(19) United States
(12) Patent Application Publication
(10) Pub. No.: US 2005/0058584 A1
(43) Pub. Date: Mar. 17, 2005

(54) Ceiling fan with sterilizing and air cleaner device

(76) Inventor: Shing-Jy Shyu, Taipei County (TW)

Correspondence Address: Shing-Jy Shyu, P. O. Box No. 6-57, Junghe, TAIPEI 235, TW

(21) Appl. No.: 10/660,501

(22) Filed: Sep. 12, 2003

(51) Int. Cl. A61L 9/00; A61L 2/00; A62B 7/08

(52) U.S. Cl. 422/305; 422/1; 422/4; 422/5; 422/24; 422/124

(57) ABSTRACT

A ceiling fan with sterilizing and air cleaner device constructed such that edges of an upper cover and a lower cover lodge into a groove of a securing annular plate which is configured with an array of protruding pieces. A cold cathode ray light tube is fitted within the ceiling fan, and a motor is further mounted within the ceiling fan; the motor connects to fan blades. A turning axle connects to a controller, and a switch is configured on the controller, wherewith the switch controls operation of the motor, brightness and turning off of the cold cathode ray light tube. Upon the ceiling fan rotating, air relatively high in a room is blown downwardly toward the floor and blown through negatively charged oxygen ions are produced by the cold cathode ray light tube, thereby enabling oxygenation of organic substances, consequently enhancing quality of indoor air.

### BACKGROUND OF THE INVENTION

(a) Field of the Invention

The present invention relates to ceiling fan, and more particularly to a ceiling fan with sterilizing and air cleaner device having edges of an upper cover and a lower cover lodge into a groove of a securing annular plate. Screws pass through screw holes to tighten down the upper cover and the lower cover to the securing annular plate. The securing annular plate is configured with an array of protruding pieces, and screws tighten down hook rings on the protruding pieces. A cold cathode ray light tube is fitted within the ceiling fan.

A motor is further mounted within the ceiling fan, the motor connects to fan blades, a turning axle connects to a controller, and a switch is configured on the controller, wherewith the switch controls operation of the motor, brightness and turning off of the cold cathode ray light tube.

Upon the ceiling fan rotating, air relatively high in a room is blown downwardly toward the floor and blown through negatively charged oxygen ions are produced by the cold cathode ray light tube, thereby enabling oxygenation of organic substances, and thereof revivification to inorganic substances, accordingly increasing dispelling of odor indoors, and eliminating germs drifting in the air, thus enhancing quality of indoor air.

(b) Description of the Prior Art

A conventional ceiling fan, apart from circulating air indoors or having lighting fixtures additionally mounted to the ceiling fan for lighting purposes, the conventional ceiling fan does not provide any other function. If air indoors is relatively unclean, upon being blown about by a fan, the air indoors becomes even more unclean. Moreover, when the air indoors is unclean considerable usage of air cleaning devices is employed to clean the air so as to improve quality of the air indoors. Furthermore, an air cleaner not only adds to extra consumption of electric power when in use, but also takes up space indoors, at the same time is non-beneficial to convection of the air indoors, thereby reducing effectiveness of the air cleaner.

Therefore, the inventor of the present invention wishes to overcome the aforementioned shortcomings of the prior art, and provide a significant contribution to resolving the technical difficulties inherent in the prior art devices.

### SUMMARY OF THE INVENTION

Referring to FIGS. 1, 2, 2-1 and 3, which show a ceiling fan with sterilizing and air cleaner device according to the present invention. The ceiling fan A is constructed to include an upper cover B, a driver C, a motor D, a cold cathode ray light tube E, a securing annular plate F, a lower cover G, fan blades H, a controller I, and a turning axle J. Main characteristics hereto are edges of the top cover B and the bottom cover G lodge into a groove F3 of the securing annular plate F, screws G1 pass through screw holes B1, F3, and G3; an array of protruding pieces F1 are configured in the securing annular plate F. The screws G1 tighten down hook rings F2 onto the protruding pieces F1, therewith securing the cold cathode ray light tube within the ceiling fan A.

Furthermore, a motor D is mounted within the ceiling fan A. The motor D connects to the fan blades H, and the turning axle J connects to the controller I. A cord controllable switch I1 is configured on the controller I, wherewith the cord controllable switch I1 controls operation of the motor D, brightness and turning off of the cold cathode ray light tube thereof.

Upon the ceiling fan rotating, air relatively high in a room is blown downwardly toward the floor and blown through light rays that have come in contact with a catalyzer tube as the light rays pass through the cathode ray light tube, enabling oxygenation of organic substances, and thereof revivification to inorganic substances, accordingly increasing dispelling of odor indoors, and eliminating germs drifting in the air, hence enhancing quality of indoor air.

Material providing protection against ultraviolet rays is employed as the material for the upper cover C and lower cover G, the small amount of ultraviolet light rays emitted from the cold cathode ray light tube E to sterilize and clean the air is controlled to be within a range that is harmless to a human body, while maintaining effectiveness to clean the air.

A topmost annular fitment B2 facilitates mounting of the ceiling fan A to a ceiling securing fixture, a driver C is secured to the upper side of the top cover B, wherewith the driver C actuates the cold cathode light tube E.

Referring to Appendix 1, wherein a sterilization test report of the present invention reveals that concentration of odor produced when the present invention is in operation indoors is within safety standard measures. Moreover, after the cold cathode ray light tube has been in continuous use for over two hours, a 90% effective reduction is achieved in the amount of bacteria indoors, and reduction in epiphyte reaches over 55%. Furthermore, this effectiveness of sterilization will not cause any harm whatsoever toward a human body.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows an elevational view according to the present invention.

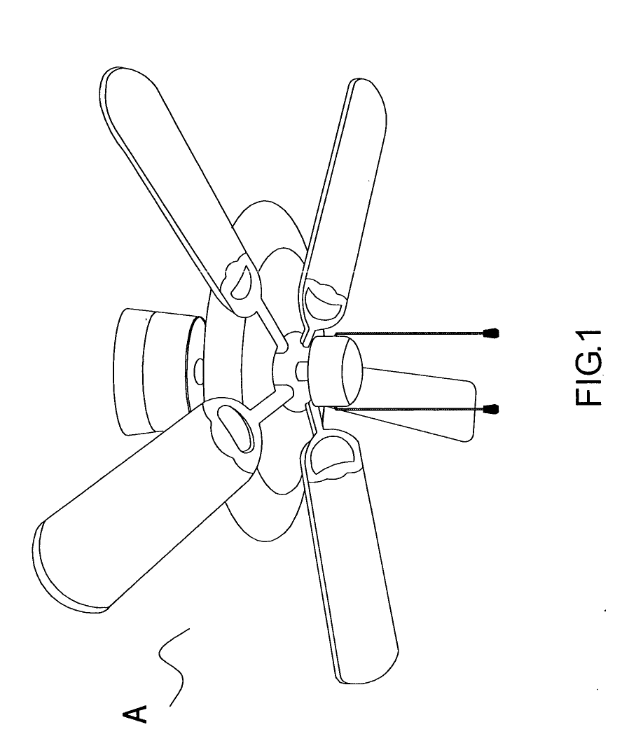

FIG. 2 shows an exploded elevational view according to the present invention.

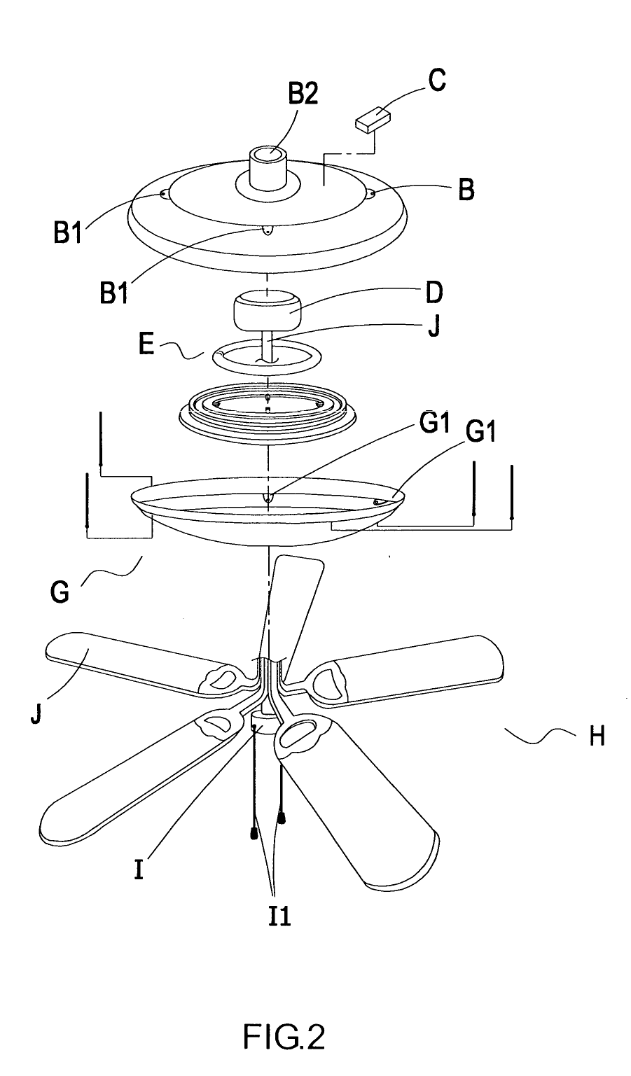

FIG. 2-1 shows a partial exploded elevational view according to the present invention.

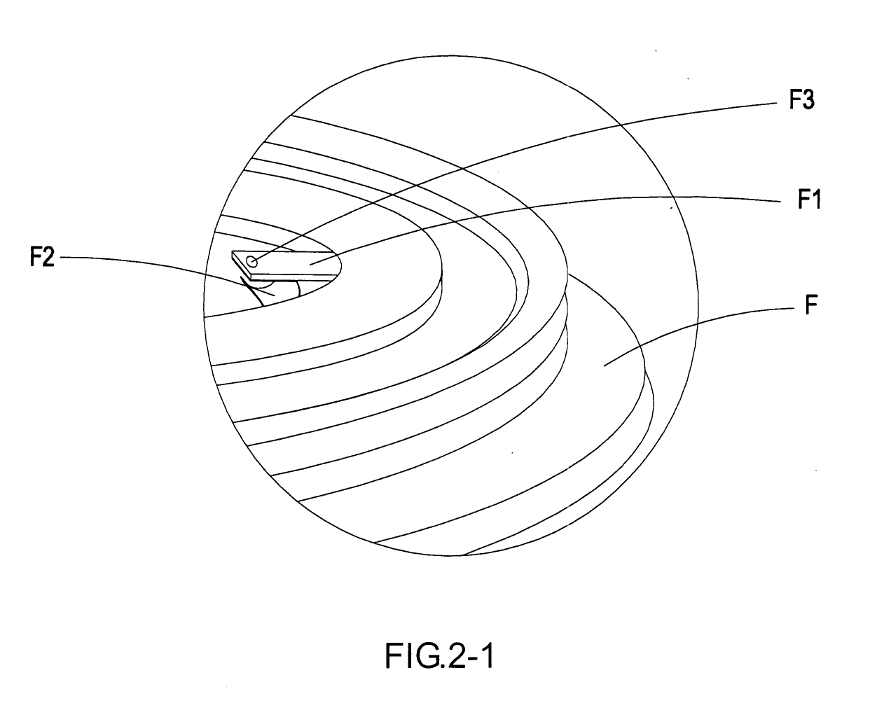

FIG. 3 shows a cross sectional view according to the present invention.

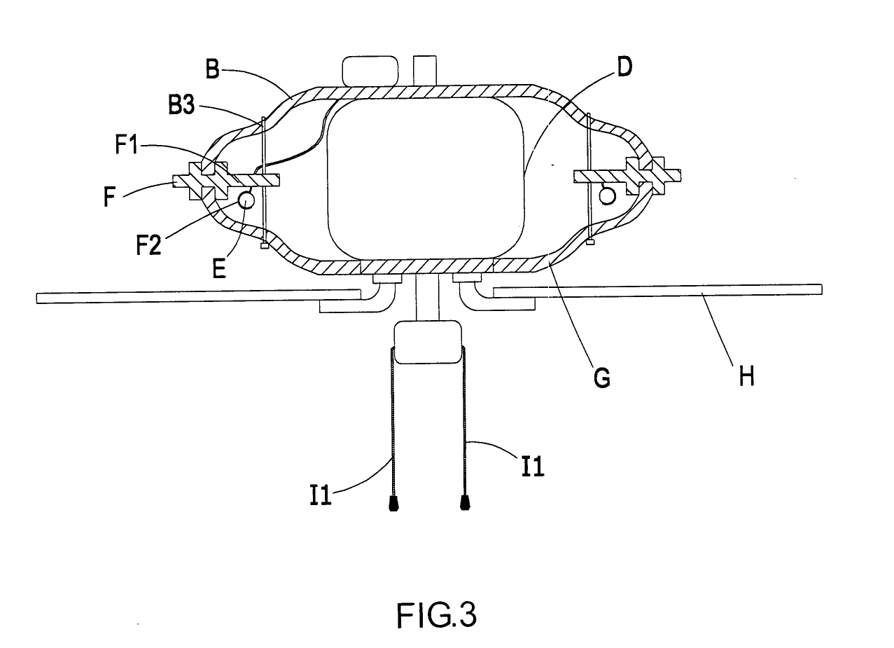

FIG. 4 shows a preferred embodiment according to the present invention.

### DETAILED DESCRIPTION OF THE PREFERRED EMBODIMENTS

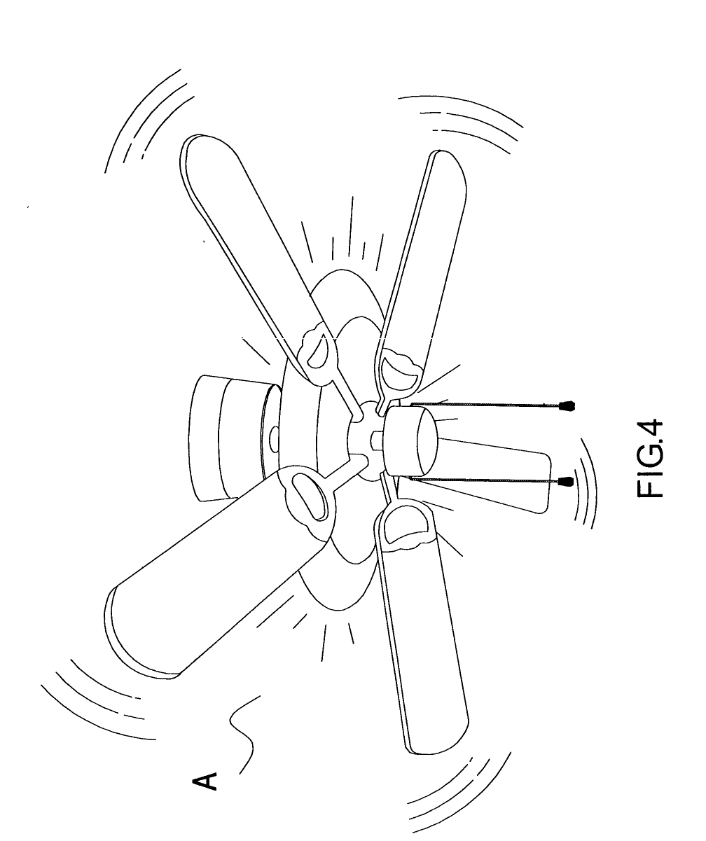

With reference top FIG. 4, wherein a preferred embodiment of the present invention is shown, comprising a ceiling fan with sterilizing and air cleaner device. Upon the ceiling fan rotating, air relatively high in a room is blown downwardly toward the floor and blown through negatively charged oxygen ions produced by a cold cathode ray light tube, thereby enabling oxygenation of organic substances, and thereof revivification to inorganic substances, accordingly increasing dispelling of odor indoors, and eliminating germs drifting in the air, thereof enhancing quality of indoor air.

In order to further demonstrate advancement and practicality of the present invention, a comparative analysis with a conventional ceiling fan is outlined below:

Conventional Shortcomings

1. Lack of functionality to sterilize air; is only adapted to having a functionality for indoor air convection.

2. An air cleaner takes up space, and only provides usage to clean air; does not include functionality for air convection and cleaning of air indoors.

3. Appliances providing functionality for cleaning air indoors and air convection are expensive, and placement of such appliances indoors takes up space.

4. A conventional air-cleaning device is excessively effective, and leakage of ultraviolet rays from indoor devices easily results in harm to a human body.

Advantages of the present invention

1. Ceiling fan includes functionality to clean air, thereby enhances quality of indoor air.

2. Utilizes wind force produced from rotating fan blades of the ceiling fan when in operation to increase efficiency of cleaning air.

3. Utilizes a cold cathode ray light tube in coordination with a casing to provide an anti-ultraviolet rays effect, and thereby achieves effectiveness of lighting and cleaning air.

4. Effectiveness of cold cathode ray light tube, along with protectiveness of the casing eliminates any harm to a human body.

5. Advancement and practicability.

6. Enhances industrial competitiveness.

In conclusion, the present invention has achieved a breakthrough in prior art technology, and assuredly accomplishing effectiveness of wished for advancement, and is easily understood by those not skilled in the art. Furthermore, the present invention has not yet been made public before this patent application.

Advancement and practicality of the present invention demonstrates compliance with application requirements of a new model patent; in conformity of such, a new model application is proposed herein.

It is of course to be understood that the embodiments described herein is merely illustrative of the principles of the invention and that a wide variety of modifications thereto may be effected by persons skilled in the art without departing from the spirit and scope of the invention as set forth in the following claims.

1. A ceiling fan with sterilizing and air cleaner device constructed to include an upper cover, a driver, a motor, a cold cathode ray light tube, a securing annular plate, a lower cover, fan blades, a controller, and a turning axle; characterized in that,
edges of the upper cover and the lower cover lodge into a groove of the securing annular plate, screws pass through screw holes and tighten down the upper cover and the lower cover to the securing annular plate, an array of protruding pieces are configured within the securing annular plate, screws tighten down hook rings onto the protruding pieces, thereby securing the cold cathode ray light tube within the ceiling fan;
the motor is further mounted within the ceiling fan, the motor connects to the fan blades, a turning axle connects to a controller, and a switch is configured on the controller, wherewith the switch controls operation of the motor, brightness and turning off of the cold cathode ray light tube;
material providing protection against ultraviolet rays is employed as the material for the upper cover and lower cover, the ultraviolet light rays emitted from the cold cathode ray light tube to sterilize and clean the air is controlled to be within a range that is harmless to a human body, while maintaining effectiveness to clean the air.
